(19) United States
(12) Patent Application Publication
(10) Pub. No.: US 2020/0011862 A1
(43) Pub. Date: Jan. 9, 2020

(54) APPARATUS AND METHOD FOR ANTIBODY DETECTION

(71) Applicant: EUROIMMUN Medizinische Labordiagnostika AG, Luebeck (DE)

(72) Inventors: Katja MORGENROTH, Hernburg (DE); Vanessa VIERTEL, Luebeck (DE); Winfried STOECKER, Gross Groenau (DE); Ulf STELLER, Buchholz (DE); Stefan GERLACH, Gross Groenau (DE); Christian MARZAHL, Erlangen (DE); Joern VOIGT, Luebeck (DE)

(21) Appl. No.: 16/502,630

(22) Filed: Jul. 3, 2019

(30) Foreign Application Priority Data
Jul. 6, 2018 (EP) EP18182267.7

(51) Int. Cl. G01N 33/543; G01N 21/76

(52) U.S. Cl. CPC G01N 33/54373; G01N 2201/10; G01N 21/76

(57) ABSTRACT

An antigen chip is described, the latter comprising a plane substrate surface; and antigen spots, which are applied in a predetermined pattern on the substrate surface and which contain a first fluorescence dye. Further, an apparatus and a method for detecting antibodies in a sample are provided. Even further, the application relates to kit for use in a method for detecting antibodies in a sample.

The present application relates to an antigen chip or antigen substrate for antibody detection and an apparatus for detecting antibodies in a sample, said apparatus comprising the antigen chip. Further, the present application relates to a method for producing an antigen chip/substrate and a method for detecting antibodies in a sample. Even further, the invention relates to kit for use in a method for detecting antibodies in a sample.

Conventionally, indirect immunofluorescence technology is used to identify antibodies in liquid samples. To this end, cells, tissue sections or cleaned, biochemically characterized substances, for example, can be used as antigen substrates. In a first incubation step, (primary) antibodies to be detected, which are contained in the samples, are bound to the solid-phase-bound antigens. In a second incubation step, (secondary) fluorescence-marked anti-human antibodies bound to these antibodies bound from the sample. Then, bound secondary antibodies can be detected in a fluorescence microscope on account of the fluorescence marking, said fluorescence marking indicating the presence in the sample of primary antibodies specific to each antigen.

WO 2012/094427 A1 discloses a method for fluorescence detection of antibodies by fluorescence detection. US 2005/0124017 discloses fluorescence imaging for detecting proteins, antibodies, drugs or other ligands in a sample, with an image being scanned. US 2004/0253640 discloses a micro-array with proteins printed thereon in order to detect a target protein by immunofluorescence. WO 2017/025954 A1 discloses an antigen chip for immunofluorescence detection, with the antigen chip being scanned for detection purposes.

WO 2012/052994 A2 discloses micro-arrays for high-throughput characterization of the immune response. WO 2012/037369 A discloses antibody detection by fluorescence detection. WO 2004/027379 A discloses a technique of rolling spheres for the purposes of detecting primary antibodies, which are bound to an antigen micro-array. WO 2000/063701 A2 discloses micro-arrays of polypeptides for the purposes of detecting antibodies, for example, wherein use is made of fluorescence detection and the micro-arrays are scanned.

US 2017/0016052 A1 discloses array systems with internal controls.

WO 2011/101487 A1 discloses a method for diagnosing disorders by way of a simultaneous detection of antibodies, which are bound to synthetic and cellular substrates, with the antibodies being detected by indirect immunofluorescence. A synthetic substrate is a microparticle or a sphere, which is coated with a cleaned native antigen were recombinant antigen; a fluorescence microscope is equipped with a camera and with a scanning system.

EP 2 362 222 discloses a method for diagnosing disorders, wherein antibodies are detected simultaneously, said antibodies being bound to cellular or tissue substrates or said antibodies being bound to synthetic substrates, such as, for instance, microparticles or spheres, which are coated with the specific antigens. Here, multicolour fluorescence microscopy is used to detect the bound antibodies, with the substrates and the bound antibodies having different fluorescence colours.

It has been observed that the conventional systems and methods for detecting antibodies do not facilitate a reliable detection of a multiplicity of antibodies in all circumstances and within an acceptable measurement time. Additionally, conventional methods require a relatively large amount of antigen or sample. Further, conventional methods only have a restricted suitability for high-throughput applications in routine medical diagnostics.

It is consequently an object of the present invention to provide systems and methods that assist a reliable detection of a multiplicity of antibodies in a liquid sample, in particular that are suitable for routine medical diagnostics and, further particularly, supply at least semi-quantitative results.

This object is achieved by the subject matter of the independent claims, which are directed to an antigen chip, to an apparatus for detecting antibodies in a sample, to a method for producing an antigen chip and to a method for detecting antibodies in a sample. Further, the object is achieved by a kit for use in a method for detecting antibodies in a sample. The dependent claims specify special embodiments of the present invention.

According to one embodiment of the present invention, an antigen chip is provided, said antigen chip comprising a plane substrate surface and antigen spots, which are applied in a predetermined pattern on the substrate surface and which contain a luminophore, a chromogenic substrate or a first dye, in particular a first fluorescence dye.

The chip can be considered to be a solid substrate, for instance a glass plate or a silicon plate, for example. The chip may also be manufactured from plastic or else be manufactured from metal. The chip may be light-transmissive or not light-transmissive, in order to assist transmitted light or reflected light illumination and detection. The antigen chip may be used in a scanner or camera system, in particular a fluorescence microscope, for the purposes of examining a sample and, in that case, it may be irradiated by illumination light or excitation light. The first dye in the antigen spot, in particular fluorescence dye, can be induced to emit light, i.e., to the first radiation (e.g., of illumination light reflected by the first dye), in particular to the first fluorescence, by the illumination light or excitation light. Further, if an antibody from the sample is bound to a certain antigen spot and if, further, the antigen spots or the entire antigen chip were incubated with secondary fluorescence-marked antibodies, the second fluorescence dye (which is bound to the secondary antibody) is preferably also induced to emit light, i.e., to the second fluorescence.

The first fluorescence may comprise an emission of light in a first wavelength range and the second fluorescence may comprise an emission of light in a second wavelength range, with the first wavelength range and the second wavelength range having only a small overlap region or substantially no overlap region, for instance an overlap region or overlap which corresponds to, e.g., less than 10%, in particular less than 5% of an integral of an emission of the first fluorescence over the wavelength. A similar definition can be provided in respect of the second fluorescence. The first and the second fluorescence can be induced by way of the same, or different, wavelengths or wavelength ranges.

The antigen spots may be printed or spotted on the plane substrate surface. To this end, use can be made of piezo-printing technology, as is already used in the prior art. By way of example, the predetermined pattern may define the arrangement, the size and/or the shape of the antigen spots. The predetermined pattern can be used to localize the spots and/or to identify the respective antigens contained in the antigen spots when carrying out a method for detecting antibodies in a sample. By way of example, antigen spots containing the same antigen may occur multiple times on the antigen chip and may be arranged along a line with a specific spot spacing and/or may have a certain shape and/or a certain sequence. Antigen spots containing a different antigen may likewise be arranged along a line; however, these may have a different (or the same) spot spacing, may have a different (or the same) shape and may have a different (or the same) size.

The antigen chip may be used in a method for detecting antibodies in a sample according to one embodiment of the present invention.

The luminophore, the chromogenic substrate or first dye, in particular fluorescence dye, may permit or facilitate localization of the antigen spots by detecting the first radiation, in particular fluorescence detection of the first fluorescence, after irradiation with illumination light, in particular excitation by excitation light. Then, the second chemiluminescence or fluorescence, which is induced by the emission of light after the excitation of the second fluorescence dye, can be detected selectively at the specific positions of the antigen spots. Then, primary and/or secondary antibodies possibly bound to the plane (naked) substrate surface away from all antigen spots can then be identified as measurement background. Then, fluorescence signals arising from the measurement background can then be subtracted from the fluorescence signals, for example, in order thus to increase the measurement accuracy.

According to one embodiment of the present invention, the antigen chip is embodied in such a way that antigen spots, which contain different antigens, contain different amounts or concentrations of the luminophore, chromogenic substrate or first dye, in particular fluorescence dye, in such a way that first radiation cast back therefrom, in particular induced first fluorescence, has different intensities or substantially the same intensity. By way of example, one (or more) row(s) of antigen spots may have a certain antigen spot spacing such that the row(s) can be distinguished from other rows of antigen spots, with one or more reference rows arising thus. A distinguishing feature of the antigen/control lines among themselves can lie in a different intensity of the first fluorescence. By way of example, first radiation cast back by antigen spots of different lines, in particular induced first fluorescence, can have substantially different intensities in order to be able to distinguish and/or identify the lines.

A localization of the antigen spots according to a uniform method can be carried out more reliably if the antigen spots, which contain different antigens, emit substantially the same intensity (or predetermined different intensities) of the first fluorescence after excitation by the excitation light. Then, it is also easier to set the exposure for recording a first image and, in particular, it is possible to dispense with making a plurality of recordings of the first image with different exposure times. Consequently, the method can be accelerated and can be carried out more reliably.

The antigens contained in the various antigen spots can detect autoimmune diseases, allergies and transmissible diseases by binding antibodies. In the present test system, this may relate, in particular, to antinuclear and extractable antinuclear antibodies from the field of connective tissue diseases.

The antigens contained in the various antigen spots may be selected and embodied to bind antibodies that are formed as a consequence of autoimmune diseases, allergies and transmissible diseases. In the present test system, this relates, in particular, to antinuclear and/or extractable antinuclear antibodies from the field of connective tissue diseases. In particular, the following antigens, for example, may be contained (alone or in any combination) in the antigen spots: RNP/Sm, Sm, Scl-70, Rib.PO, Jo-1, SS-A, SS-B, dsDNA, nucleosomes/chromatin, Cenp-B, RNP A,C,68 kDa, Ro-52, Ku, histones, DFS70. The diagnosable connective tissue diseases may contain at least one of the following forms: SLE (Systemic lupus erythematosus), PM, DM (Myositis), SS (Sjögren's syndrome), CREST syndrome (limited cutaneous form of systemic sclerosis IcSSc), PSS (progressive systemic sclerosis), MCTD (mixed connective tissue disease, sharp syndrome), AID (autoimmune induced disease). Consequently, various diseases can be diagnosed.

The expression “chemiluminescence”, as used herein, relates to a chemical reaction in which energy is specifically guided to a molecule, causing the latter to be electronically excited and subsequently release a photon, as a result of which visible light is emitted. Thermal energy is not required for this reaction. Consequently, chemiluminescence contains a direct conversion of chemical energy into light energy. Preferably, chemiluminescence arises via the reaction of a luminophore with other compounds. These reactions may be catalysed by enzymes. In further preferred embodiments of the invention, the luminophore is selected from the group consisting of luminol and derivatives thereof, acridine and derivatives thereof and luciferins. These compounds can be induced to exhibit chemiluminescence by various enzymatic reactions. The corresponding compounds and reactions are known in the prior art. In further preferred embodiments of the invention, the luminophore is luminol or a derivative thereof, wherein the light is emitted (chemiluminescence) by way of a reaction catalysed by peroxidase, in particular horseradish peroxidase. In alternative embodiments of the invention, the luminophore is acridine or a derivative thereof, in particular acridinium ester or acridinium sulfonamide, wherein the light is emitted (chemiluminescence) by way of a reaction catalysed by phosphatase, in particular alkaline phosphatase (AP). In further alternative embodiments of the invention, the luminophore is a luciferin, in particular D-luciferin, wherein light is emitted (chemiluminescence) by a reaction catalysed by luciferase.

A “chromogenic substrate” within the meaning of the present invention is a reagent that can be used for measuring enzyme activities. The chromogenic substrate is modified by enzymatic activity in such a way that a direct or indirect product of the enzymatic reaction can be quantified (photometrically). By way of example, the chromogenic substrate consists of an oligopeptide, to which an azo dye, e.g., paranitroaniline, is coupled. The peptide imitates the interface at which the enzyme to be examined splits its physiological substrate. The dye is released by the enzyme action. The colour development can be quantified photometrically and the enzyme activity can be ascertained by way of the calibration. However, the chromogenic substrate can also be 5-bromo-4-chloro-3-indolyl phosphate (BCIP) and/or nitro blue tetrazolium chloride (NBT). By way of example, a chromogenic substrate can be detected colorimetrically.

According to one embodiment of the present invention, the antigen chip is embodied in such a way that antigen spots of the same antigen are arranged in a line in each case, in particular with a predetermined spot spacing, wherein lines assigned to different antigens have, in particular, a predetermined line spacing, wherein, further, the line spacing of two lines is greater than the spot spacing within a line. However, the spacing between two lines may alternatively also be smaller than the spot spacing within the line.

An evaluation system of an apparatus for detecting antibodies in a sample may be embodied, for example, to determine a line spacing between two lines of antigen spots and/or a spot spacing of antigen spots within a line. The spot spacing may be specific to antigen spots containing a certain antigen. Therefore, the antigen contained in the antigen spots of the examined line can be deduced from the determined spot spacing. The line spacing of a first line of antigen spots from a second line of antigen spots can likewise be determined in order to determine those antigens or that antigen contained in the antigen spots of the second line, adjacent to the first line, for which the antigen contained in the antigen spots has already been determined. A certain line spacing and a certain spot spacing can be combined in order to determine a reliable localization of the antigen spots and/or an assignment of the antigen spots to certain antigens.

The antigen spots may have different forms; in particular, they may be substantially circular and substantially have different sizes, in particular a diameter that is substantially the same or different. Consequently, it is possible to use conventional spotting techniques to apply the antigen spots to the substrate surface.

According to one embodiment of the present invention, the antigen chip is embodied in such a way that, further, at least one, two, in particular at least three spots or lines of antigen spots (also referred to as calibration spots or internal calibrator) are applied to the substrate surface, said spots or lines of antigen spots containing a primary antibody (e.g., IgG) in identical or different amounts or concentrations. Following incubation with a fluorescence-marked secondary antibody (which is marked by the second fluorescence dye), a second fluorescence can also emanate from the calibration spots containing the primary antibody, to be precise in various intensities in accordance with the various amounts or concentrations of the primary antibody. As a result, a calibration of an evaluation system can be carried out by evaluating the intensities of the second fluorescence in order to permit a quantification of antibodies bound to other antigen spots. By way of example, the various amounts or concentrations may be predetermined or known. The intensities of the second fluorescence, detected by the various calibration spots, can be assigned to the respective amounts or concentrations and extrapolation or interpolation can be carried out to yield intermediate concentrations or concentrations that are greater or smaller than the concentrations contained in the calibration spots. Consequently, the antigen chip may support quantitative or at least a semi-quantitative analysis of antibodies in a sample. An external calibrator (in the form of calibrator serum) can be used for precise quantification.

According to one embodiment of the present invention, the antigen chip is embodied in such a way that, further, at least one line, in particular a line of an antigen with a smaller dot spacing in comparison with the dot spacings of the other lines of the antigen chip, is applied to the substrate as a positive control. In one embodiment, this line can be developed as a continuous line. Expressed differently, the antigen chip according to one embodiment of the present invention is developed in such a way that spots with different spacings, right down to a continuous line, are embodied on the substrate.

According to a further embodiment, antigen spots which, for example, contain the same antigen may be arranged along a line, with the spot spacing thereof being so small that the various spots are in contact with one another and are perceived as a continuous line. Thus, the antigen chip of the present invention comprises chips which consist only of spots, only of lines or of a combination of spots and lines.

The positive control (e.g., anti-human IgG) can be used to determine whether the relevant antigen chip has even been incubated with the sample. By way of example, every expected sample may contain one or more specific antibodies in all cases, even if no pathological condition is present. The position or the localization of the positive control, too, can be determined by the evaluation system and can be used, in particular, to determine the orientation of the antigen chip. By way of example, the unique spot spacing within the lines and/or the arrangement of the lines with different spot spacings from one another may serve to orient the antigens on the chip. Hence, an analysis of antibodies can be carried out more reliably.

The first fluorescence dye can fluoresce, e.g., in a red wavelength range, for example in a range between 550 nm and approximately 800 nm with a maximum between 650 nm and 700 nm. To this end, use can be made, for example, of the first fluorescence dye DY521XL. The excitation of the first fluorescence dye can be implemented in frequency range of, for example, between 450 nm and 650 nm with a maximum between 500 nm and 540 nm, for example. The second fluorescence dye can be excited in substantially the same wavelength range as the excitation of the first fluorescence dye. As a result, only a single illumination light source, for example, which may have a relatively narrow-band embodiment, is required to produce the excitation light in an apparatus for detecting antibodies in a sample. This advantageously allows the excitation light to be filtered out before the first fluorescence is detected by a first camera and the second fluorescence is detected by a second camera or both fluorescence signals are detected by the first or the second camera (e.g., after a filter change), without detecting substantially disturbing excitation light.

According to one embodiment of the present invention, the substrate surface is microstructured for focusing purposes. The microstructuring can be implemented by a surface treatment (e.g., roughening) and can simplify focusing on the substrate surface. Various methods for microstructuring are known in the prior art, for example injection moulding, hot stamping or imprint methods. Various microstructured markers can be applied to the surface; these can also be used to identify the antigen chip, to identify the sample, etc.

The antigen substrate (the chip) comprises an area with the size of between 1 mm×1 mm and 2 mm×4 mm, in particular.

According to one embodiment none of the antigen spots comprises a gel or a gel spot or gel pad, in particular a (3D) polymer gel and/or a polyacrylamide gel, in which the antigens are bound. According to one embodiment, the antigen spots merely comprise the first dye, chromogenic substrate or luminophore, the antigen and (dried) buffer without polymer and/or have a thickness of between 0.1 μm and 5 μm.

Reference is made to the fact that features that were described, explained or provided in conjunction with an antigen chip, either individually or in any combination, likewise can be applied, either individually or in any combination, to an apparatus for detecting antibodies in a sample, to a method for producing an antigen chip and also to a method for detecting antibodies in a sample, and vice versa, according to embodiments of the present invention.

According to one embodiment of the present invention, an apparatus for detecting antibodies in a sample is provided, comprising: an antigen chip according to one of the preceding embodiments; and a scanner or camera system with an illumination source (e.g., a fluorescence microscope), which is embodied to produce illumination light, in particular excitation light at a first wavelength, and which is arranged to illuminate, in particular irradiate, the antigen chip; a first camera, which is embodied to record a first image by detecting first radiation emanating from the sample caused by the illumination light, in particular first fluorescence induced by the excitation light, and/or embodied to record a second image by detecting chemiluminescence emanating from the sample or second fluorescence induced by the illumination light, in particular excitation light.

The scanner and/or camera system, in particular the fluorescence microscope (55), can further comprise a second camera, which is embodied to record a second image by detecting chemiluminescence emanating from the sample or second fluorescence induced by the illumination light, in particular if the first camera cannot provide the detection of the second fluorescence.

The scanner or camera system for capturing chemiluminescence or fluorescences, e.g., the fluorescence microscope, may comprise an object holder in which the antigen chip can be held. Further, the fluorescence microscope may comprise an objective lens, which may include one or more lenses and by means of which it is possible to produce a magnified image of the antigen chip or of at least a part of the antigen chip when the illumination source illuminates the antigen chip. Further, the fluorescence microscope may comprise at least one beam splitter (e.g., a semi-transmissive mirror) to direct one part of the (fluorescence) light emanating from the antigen chip on the first camera and direct another part on the second camera. The apparatus can be embodied to record the first image at the same time as or with a time offset from the second image. The illumination source can direct the excitation light on the antigen chip while the first image is recorded and while the second image is recorded. The excitation light induces the first fluorescence dye to emit the first fluorescence. In preferred embodiments, it also induces a second fluorescence dye (which is bound to a secondary antibody, in particular) to emit second fluorescence. Consequently, the apparatus need only have a single illumination source in order to carry out a method for detecting antibodies in a sample using the antigen chip as described above. By way of example, the illumination source can comprise a laser, an LED, in particular a cLED. Other light sources are possible.

Further, the apparatus may comprise a first filter, which is arranged in a first beam path upstream of the first camera and which is embodied to substantially eliminate excitation light and/or the light of the second fluorescence, and, in particular, further comprise a second filter, which is arranged in a second beam path upstream of the second camera and which is embodied to substantially eliminate excitation light and/or the light of the first fluorescence. The first filter can pass a first wavelength range about a maximum of the first fluorescence but substantially eliminate a second wavelength range around the maximum of the second fluorescence. Using this, it is possible to reliably carry out a localization of the antigen spots. Conversely, the second filter can substantially pass a second wavelength range around the maximum of the fluorescence of the second fluorescence but the light of the first wavelength range around a maximum of the first fluorescence is substantially eliminated or attenuated. Using this, it is possible to reliably determine the number of antibodies which are bound to the various antigen spots or to the antigens contained therein. By way of example, the first beam path or the first fluorescence may contain red light and it is sometimes also referred to as red channel. By way of example, the second beam path or the second fluorescence may contain green light and it is sometimes also referred to as green channel. Light with different wavelengths are comprised in the first beam path and the second beam path, or in the first fluorescence and in the second fluorescence, in other embodiments for as long as, however, the corresponding wavelength ranges are sufficiently separated from one another or only have a small overlap. The system may exploit a (single) excitation wavelength (or excitation wavelength range) for both fluorescence dyes. According to another embodiment, the two fluorescence dyes are excited at different wavelengths or wavelength ranges (e.g., using different light sources).

In a preferred embodiment of the apparatus, the latter is embodied in such a way that fluorescence can be induced and detected, with the first and second fluorescence being inducible by way of the same wavelength. In further preferred embodiments, the first fluorescence is produced by exciting DY-521-XL and the second fluorescence is produced by exciting fluorescein isothiocyanate (FITC).

According to one embodiment of the present invention, the apparatus may further comprise an evaluation system which is embodied to determine positions of the antigen spots on the basis of the first image, to assign positions of the antigen spots to respective antigens on the basis of the predetermined pattern and to recognize antibodies bound to antigen spots, in particular at least semi-quantitatively, on the basis of the second image.

The first image may also be referred to as a red image and the second image may also be referred to as a green image. Here, the first camera and also the (optional) second camera may each comprise a light-sensitive field (e.g., a two-dimensional field) of light-sensitive elements, for instance photodiodes, or may be embodied as a CMOS sensor field, for example. Here, the first camera and also the second camera may have a suitable imaging optical unit, for example.

The evaluation system can be embodied to carry out image processing. By way of example, the evaluation system can be software-controlled and can have a processor embodied to carry out the software. The positions of the antigen spots determined by the evaluation system can be correlated with positions of the predetermined pattern in order, for example, to determine the orientation and magnification on the microscope and to match these to one another. By way of example, the evaluation system can be embodied to integrate (or average) intensities in the second image over regions in which an antigen spot was determined in the first image. Then, background fluorescence/background intensities, i.e., intensities of the regions of the second image which lie outside of antigen spots determined in the first image, can be subtracted from these integrated or average intensities, for example. Consequently, the evaluation system can be embodied to evaluate the second image in order to determine intensities of the second fluorescence emanating from the individual antigen spots and, in particular, to average intensities over a plurality of antigen spots containing the same antigen. Averaging intensities emanating from antigen spots containing the same antigen may increase the measurement accuracy.

According to one embodiment of the present invention, the evaluation system is further embodied to determine intensities of second fluorescence emanating from at least one two or three or more calibration spots or calibration spot rows in the second image in order to carry out a calibration, wherein the calibration spots or calibration spot rows contain a primary antibody (e.g., IgG) in different amounts or concentrations. The amounts or concentrations of the primary antibody in the calibration spots may be known in advance or predefined. By detecting the intensities emanating from the calibration spots or calibration spot rows, the evaluation system can derive a dependence of concentration or number and obtained intensity. By way of example, the number of antibodies from various samples can be determined relative to one another for a given patient over the course of a disorder. Conversely, the concentration or number of antibodies bound to a certain antigen spot can be deduced from a detected intensity of the second fluorescence, which emanated from this antigen spot, in order to facilitate a quantitative or at least a semi-quantitative evaluation.

According to one embodiment of the present invention, the evaluation system is further embodied to recognize at least one line, in particular a line with the smaller dot spacing in comparison with the dot spacings of the other lines of the antigen chip, of an antigen (as a positive control) in the first image in order to determine an orientation of the antigen chip.

According to one embodiment of the present invention, a method for producing an antigen chip for indirect immunofluorescence diagnostics is provided, including applying antigen spots, which contain a luminophore, a chromogenic substrate or a first dye, in particular a first fluorescence dye, on a plane substrate surface of a substrate in a pattern that has been predetermined for each antigen, and fragmenting the substrate (e.g., into pieces at arbitrary points and/or breaking curves; i.e., a positionally inaccurate fragmentation) in order to obtain a plurality of antigen chips. Since the fragment is larger than one array in preferred embodiments, at least one complete set of spots for an array is present on a chip/substrate. Fragmenting the substrate into a plurality of pieces in order to obtain a plurality of antigen chips may be implemented with a certain inaccuracy in respect of the positioning of break lines or cut lines. Absolute positioning of the antigen spots relative to the edges of the antigen chip need not be required in order to carry out a method for detecting antibodies since the positions of the antigen spots are determined during the detection method without having a precondition of an absolute predetermined positioning of the antigen spots. In preferred embodiments with at least two reference antigen spots or reference antigen spot rows, it is possible to rotate the antigen substrate through 180° (e.g., during the production process) and carry out a unique assignment of the antigen spots on account of the at least two reference antigen spots or reference antigen spot rows with distinguishable spot spacings/patterns, for example. Consequently, relatively inaccurate production methods or fragmentation methods can be used, simplifying outlay, costs and complexity of the production.

The antigen spots can be applied by means of a piezoelectric micro-metering appliance and the substrate surface can be a surface of a glass substrate or of a glass substrate coated with a membrane and/or film, in particular. Hence, the production can be carried out using conventional base materials and production machines.

According to one embodiment of the present invention, a method for detecting antibodies in a sample is provided, in particular by indirect immunofluorescence, wherein the method includes: providing antigen spots, which contain a luminophore, chromogenic substrate or first dye, in particular a first fluorescence dye, and which are arranged on a plane substrate in a predetermined pattern, incubating the antigen spots with the sample; incubating the antigen spots with a secondary antibody, which is marked by a second luminophore, a second chromogenic substrate, enzyme or second fluorescence dye; illuminating the antigen spots with illumination light, in particular excitation light, in order to trigger a first radiation (e.g., illumination light scattered back from the spots, in particular the first dye), in particular in order to excite a first fluorescence of the first fluorescence dye (e.g., red channel) and to excite a second fluorescence of the second fluorescence dye (e.g., green channel), detecting the first radiation, in particular the first fluorescence, by recording a first image with a first camera and recording a second image with the first or second camera in order to determine positions of the antigen spots; assigning the positions of the antigen spots to the respective antigens on the basis of the predetermined pattern; and detecting the second chemiluminescence or fluorescence in the second image in order to recognize (in particular quantitatively) antibodies bound to antigen spots.

In a preferred embodiment of the method, the first and second fluorescences are inducible by way of the same wavelength. In further preferred embodiments, the first fluorescence is produced by exciting DY-521-XL and the second fluorescence is produced by exciting fluorescein isothiocyanate (FITC).

The method can be carried out by means of an apparatus for detecting antibodies according to one embodiment. Further, the second fluorescence dye can be selected in such a way that it can be induced by excitation light that is suitable for exciting the first fluorescence dye, but it fluoresces in a second wavelength range that is substantially different from a first wavelength range in which the first fluorescence dye fluoresces. By way of example, the sample can be a serum, a plasma or any other sample material, for example of human origin. The first image can be assembled from a plurality of first partial images (e.g., four partial images), which image different portions of the antigen chip. The second image can likewise be assembled from a plurality of partial images (e.g., four partial images), which have been successively recorded from different portions of the antigen chip. In other embodiments, only a single first image of the entire antigen chip is recorded in each case and only a single second image of the entire antigen chip is recorded.

In preferred embodiments, the method for detecting antibodies may comprise a so-called “cut-off calibrator” and the measurement thereof. The cut-off calibrator is used as a reference for distinguishing between “positive” and “negative” signals. The type of cut-off calibrator can be matched to the given parameters of the detection system and can be modified, for example, in a manner corresponding to the type of antigen, antibody and the dye, in particular the fluorescence dye. In further preferred embodiments, the cut-off calibrator can be an external calibrator. This can be a calibration sample, the concentration of which is approximately level with the cut-off of the sample to be measured, for example. This calibration sample can be incubated in parallel on a separate array (of the same array batch) and can be related in relative fashion to the intensities obtained by the samples. In alternative embodiments, the cut-off calibrator can be in internal calibrator, with the calibrator in this case being a spot comprising corresponding calibration material on the antigen chip according to the invention, such as a human IgG spot, for example.

According to one embodiment of the present invention, a kit is provided for use in a method for detecting antibodies in a sample, in particular by indirect immunofluorescence. The kit comprises an antigen chip according to one of the embodiments described above and a probe, in particular a secondary antibody, which is marked by a second luminophore, second chromogenic substrate, enzyme or second fluorescence dye. The first and the second fluorescence dye of the kit may be induced by substantially the same wavelength range (although this is not mandatory). In preferred embodiments, the first and the second dye each are a fluorescence dye and the first and the second fluorescence are inducible by way of the same wavelength, with the emission spectra, in particular the emission maxima, differing. In preferred embodiments, the emission maxima of the two fluorescence dyes to be induced in comparable fashion are spaced apart by at least 5 nm, at least 10 nm, at least 20 nm, at least 25 nm, at least 30 nm, at least 35 nm, at least 40 nm, at least 45 nm, at least 50 nm, at least 55 nm or at least 60 nm. In further preferred embodiments, the first fluorescence is produced by exciting DY-521-XL and the second fluorescence is produced by exciting fluorescein isothiocyanate (FITC).

Embodiments of the present invention facilitate localizing a spot of an array by way of a first (fluorescence) dye, chromogenic substrate or luminophore and exploiting this localization for measuring the signal, for example of a second fluorophore.

Rather than comprising one antigen chip, an object carrier may also comprise a multiplicity of antigen chips (in particular, 2, 3, 4, 5, 6, 7, etc. antigen chips) and the antigen spot properties described herein for an individual antigen chip and the predetermined pattern of the antigen spots can be transferred to the multiplicity of antigen chips (for each individually or in the entirety thereof). By way of example, the first antigen chip on an object carrier comprising two antigen chips according to the invention may comprise an incubation control and the second antigen chip may comprise a calibration spot line. Thus, the spot arrangements and properties described for one antigen chip herein may be implemented by the multiplicity of antigen chips in the entirety thereof, without each individual antigen chip of the multiplicity of antigen chips individually implementing these spot arrangements and properties.

Further, embodiments of the present invention facilitate (separately or in combination) the delimitation of one or more anti-spot rows from further spot rows of an antigen chip (e.g., by way of the spacing of the individual spots) in order to be able to unambiguously determine the orientation of the chip and consequently also the type of antigen spot rows (e.g., if the chip contains spot rows with various antigens).

An antigen chip can comprise at least one antigen spot row or at least one calibration spot row, preferably at least two antigen spot rows or calibration spot rows, which can be distinguished from the other spot rows by at least one of the following marking options:
type of dye (e.g., in respect of the reflected or excited emission);
intensity (amount or concentration) of the dye;
spacing of the antigen spot row from other antigen spot rows (spacing L in FIG. 2A);
spacing of the antigen spots within a row (spacing d in FIG. 2A);
form (e.g., circular, rectangular, square, oval, embodiment as a continuous line) of the antigen spots; and/or circumference (or extent/diameter) of the antigen spots.

The technical advantage that can be obtained thereby is that the production of the antigen chip can tolerate less accurate positioning of the antigen spots or less accurate fragmentation (e.g., breaking of the glass) since the identification and localization of the antigen spots, when said antigen chip is used in a scanner or camera system, is implemented by evaluating the first image without the necessity of predetermined positioning of the antigen spot and/or of the substrate of the antigen chip relative to the camera system.

Embodiments of the present invention are now explained with reference to the attached figures. The invention is not restricted to the illustrated or described embodiments.

### BRIEF DESCRIPTION OF DRAWINGS

FIG. 1 illustrates a plan view of an object carrier with a plurality of antigen chips according to an embodiment of the present invention;

FIG. 2A illustrates a schematic representation of an antigen chip with antigen spots in a predetermined pattern according to one embodiment of the present invention;

FIG. 2B illustrates a first image for localizing antigen spots, which was evaluated and recorded according to one embodiment;

FIG. 3 schematically illustrates an apparatus for detecting antibodies in a sample according to one embodiment of the present invention;

FIGS. 4 and 5 illustrate a first image and a second image, respectively, as are recorded and evaluated according to embodiments of the present invention;

FIG. 6 illustrates an emission spectrum of an illumination source of an apparatus according to one embodiment of the present invention;

FIG. 7 illustrates an excitation spectrum and an emission spectrum of a dye, which is used in embodiments of the present invention; and

FIG. 8 illustrates emission spectra of two fluorescence dyes, as may be used in embodiments of the present invention.

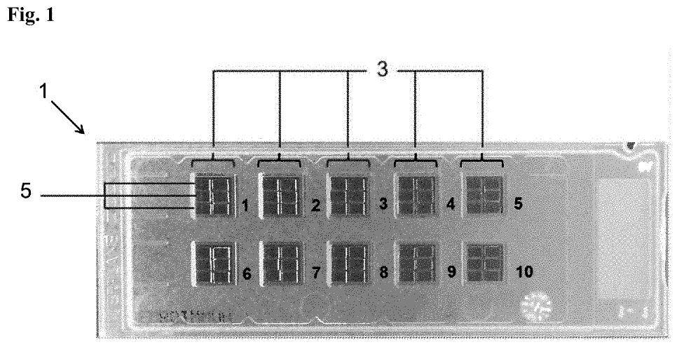

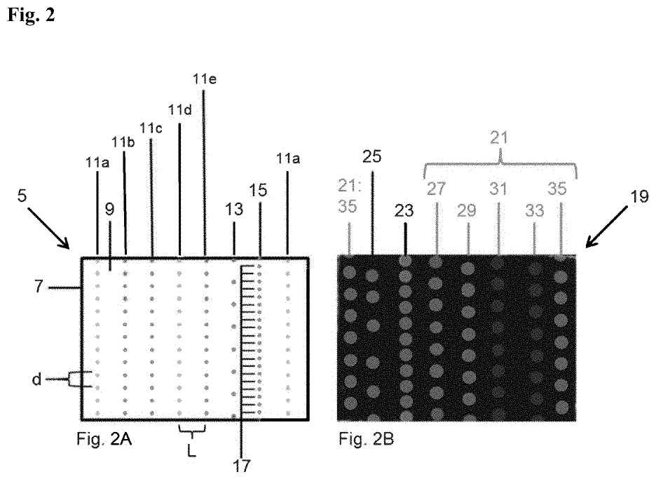

The object carrier 1 illustrated in a plan view in FIG. 1 comprises ten object carrier fields 3, which are arranged in two rows with five fields 3 each and which are numbered 1 to 10. Each field 3 in FIG. 1 comprises six antigen chips 5 (it could be 1, 2, 3, 4 or 5 antigen chips 5 in other preferred embodiments), which are provided according to one embodiment of the present invention. By way of example, an antigen chip 5 is illustrated schematically in FIG. 2A. The antigen chip 5 comprises a substrate 7 which has a plane substrate surface 9. Antigen spots 11a, 11b, 11c, 11d, 11e are applied to the substrate surface 9 in a certain pattern, with the antigen spots 11a, 11b, 11c, 11d, 11e each containing a first fluorescence dye and with the line 11a being applied in a twofold embodiment on the antigen chip. A single embodiment of the line 11a is also possible in other embodiments. Further, the line 13 with a wider dot spacing in comparison with the lines 11a, 11b, 11c, 11d, 11e, is applied to the substrate surface 9, said line allowing the determination of whether the antigen chip 5 was incubated with a sample. Further, a line 15 has been applied to the antigen chip 5, said line being formed by a plurality of calibration spots 17 containing a primary antibody in the same or different amounts or concentrations.

The antigen spots 11a are arranged along a line and each contain the same antigen. The antigen spots 11b, 11c, 11d and 11e are likewise each arranged along a line and contain the same antigen in each line. The spots arranged in the lines for an antigen may have more or fewer spots in an antigen chip 5 than what is imaged, for example between two and twenty spots for each antigen, in particular between four and ten spots for each antigen. Antigens in one line of antigen spots may be contained with the same or different concentrations or amounts in the respective antigen spots. A line of antigen spots can be characterized by spot spacing d, and by a spot size and/or a spot form. The spot spacings d for lines that each contain different antigens can be different or substantially the same. The antigen spots 11a, 11b, 11c, 11d, 11e arranged in lines are spaced apart from one another by certain spacing L. The distance between various lines of various antigens may vary. The forms of the antigen spots of the spots 11a, 11b, 11c, 11d, 11e are substantially circular. The pattern with which the various antigen spots 11a, 11b, 11c, 11d, 11e are applied, in particular spotted, to the substrate surface 9 is provided or defined by the spot spacing d (which can differ or be the same in each of the lines) and the line spacing L (which can likewise differ or be the same between various line pairs) and also by the form and/or size of all antigen spots.

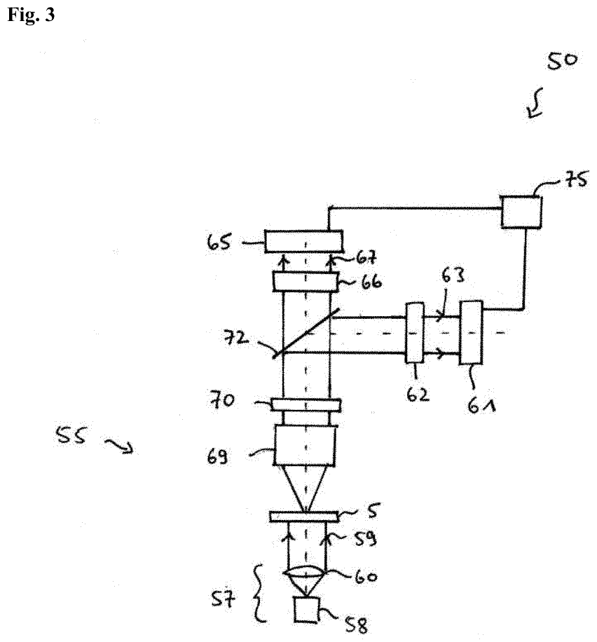

An evaluation system (as is illustrated in FIG. 3, for example) can know the geometry of the pattern with which the antigen spots are applied to the substrate surface and can also know the occupancy of the individual spots with antigens. Then, recognizing this pattern in recorded images may allow an assignment of the antigen spots to certain antigens and consequently allow an assignment of bound antibodies to certain antigens.

FIG. 2B schematically illustrates an image 19, recorded by an apparatus (e.g., from 4 partial images), of the antigen chip 5 illustrated in FIG. 2A, for example. The first fluorescence which emanates from the emission of light by the first fluorescence dye is captured for the purposes of recording the image 19 (e.g., a first image). As explained above, the first fluorescence dye is contained in all antigen spots 11a, 11b, 11c, 11d, 11e and also in the positive control line 13 and in the calibration spots 17. The intensities of the first fluorescence recorded in the image 19 are represented by shades of grey, with brighter shades of grey indicating a higher detected intensity. The bright spots or regions 21, 23 and 25 are referred to as first fluorescence spots below. As is evident from FIGS. 2A and 2B, the first fluorescence spots 21, 23 and 25 appear exactly at the positions at which the antigen/antibody spots 11a, 11b, 11c, 11d, 11e and the lines 13 and 15 are applied to the substrate surface 9. By way of a comparison with the pattern known in advance, an evaluation system recognizes that, for example, the column 35 of first fluorescence spots 21 originates from the first fluorescence emanating from the antigen spots 11e. Further, the evaluation system determines that the columns 27, 29, 31, 33, 35 originate from fluorescence emanating from the antigen spots 11a, 11b, 11c, 11d and 11e, respectively. Hence, the evaluation system is able to implement an assignment to a determined antigen or the positive control or the calibration for each region of the image 19.

In a schematic representation, FIG. 3 illustrates an apparatus 50 for detecting antibodies in a sample according to one embodiment of the present invention. Here, the apparatus 50 comprises an antigen chip 5, for example the antigen chip 5 illustrated in FIG. 2A. Further, the apparatus comprises a fluorescence microscope 55, which has an illumination source 57 embodied to produce excitation light 59 and arranged to irradiate the antigen chip 5 with the excitation light 59. In detail, the illumination source 57 comprises an LED 58 and an imaging optical unit 60 to completely or at least partly illuminate the antigen chip 5. Further, the fluorescence microscope 55 comprises a first camera 61, which is embodied to record a first image (e.g., the image 19 in FIG. 2B) by detecting first fluorescence 63 that emanates from the sample and has been induced by the excitation light 59. Further, the fluorescence microscope 55 comprises a second camera 65, which is embodied to record a second image (see FIG. 5, for example) by detecting second fluorescence 67 that emanates from the sample and has been induced by the excitation light 59. The fluorescence microscope further comprises an objective lens 69 (with one or more lenses), which is embodied to produce a magnified image of the antigen chip 5.

After the antigen chip 5 has been incubated with a sample potentially containing antibodies that bind to one or more of the antigen spots, the sample is incubated with a second fluorescence-dye-marked antibody. The excitation light 59 induces both the first fluorescence dye (contained in the antigen spots) and the second fluorescence dye (bound to a secondary antibody, for example) to excite a first and second fluorescence, the light of which simultaneously passes through the objective lens 60. An excitation light filter 70 is arranged downstream of the objective lens in order to filter out substantial parts of the excitation light 59 but in order to substantially pass light of the first fluorescence 63 and also light of the second fluorescence 67. Thereupon, the light of the first fluorescence and second fluorescence strikes a semi-transmissive mirror 72, which reflects a part so that the latter strikes a first filter 62 and passes therethrough. The first filter 62 is arranged upstream of the first camera 61 in a first beam path and embodied to substantially eliminate the excitation light 59 and light of the second fluorescence 67. Consequently, substantially only the light 63 of the first fluorescence strikes the first camera 61. Part of the fluorescence light is transmitted by the semi-transmissive mirror 72 and passes through a second filter 66, which is arranged upstream of the second camera 65 in a second beam path and embodied to substantially eliminate excitation light 59 and the light of the first fluorescence 63. Consequently, substantially only the light 67 of the second fluorescence strikes the second camera 65.

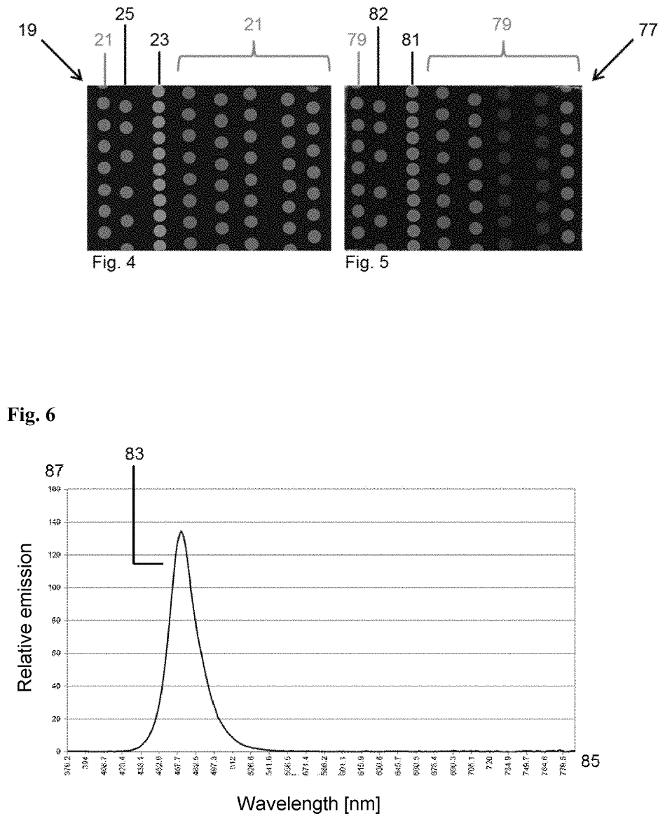

The apparatus 50 further comprises an evaluation system 75, which receives first images and second images recorded by the first camera 61 and the second camera 65, respectively, and which is embodied to evaluate said images. To this end, positions of the antigen spot are determined on the basis of the first image (e.g., image 19 in FIG. 2B) and the positions of the antigen spots are assigned to antigens in each case on the basis of the predetermined pattern. Proceeding from the first fluorescence spots 21, 23 and 25 localized in the first image, it is possible to assign intensities recognized in the second image (e.g., FIG. 4 or 5) to certain antigens in order thus to qualitatively detect or at least semi-quantitatively recognize bound antibodies.

FIGS. 4 and 5 illustrate examples of a first image 19 and of a second image 77, which are obtained and processed by the evaluation system 75. The first fluorescence spots 21, 23 and 25 recognized by the evaluation system 75 in the first image are determined in terms of their position and size. The positions and sizes of the first fluorescence spots determined in the first image 19 are then transferred to the second image 77 in order to define regions in which antigen spots with known antigens were originally present on the antigen chip. These regions then contain second fluorescence spots 79, 81, 82, the intensities of which (i.e., intensity of light of the second fluorescence) are assigned to certain antigens by the evaluation system 75. In order to be able to implement a quantification, the evaluation system can be further embodied to determine the second fluorescence emanating from at least two, in particular at least three calibration spots in the second image intensities in order to implement a calibration. The calibration spots 17 of the antigen chip 5 in FIG. 2A may contain, for example, a primary antibody (e.g., IgG) in the same or different amounts or concentrations. The secondary antibodies will also bind to these primary antibodies after incubation with the secondary antibody that has been marked with the second fluorescence dye, and so a second fluorescence will also emanate from these regions, said second fluorescence depending on the original concentration of the primary antibody.

In particular, an automatic spot identification in the red channel and an assignment of the different antigens can be carried out by the evaluation system. An automatic qualitative or quantitative evaluation of the antigen reactivity can be implemented in the green channel. A semi-quantitative evaluation can be carried out by means of standardized control serums and a specification of the fluorescence intensity values for each antigen. It is also possible to create a calibration curve for the green fluorescence on the basis of human IgG dots.

Embodiments of the present invention also provide a production method for an antigen chip, wherein antigen spots containing a fluorescence dye are applied to a plane substrate surface of a substrate in a pattern that has been predetermined for each antigen and the substrate is subsequently fragmented (without requiring exact positioning of the separating lines) in order to obtain a plurality of antigen chips. The antigens can be implemented using a piezoelectric micro-metering appliance, for example on glass or glass-coated membrane/film. A patient sample (e.g., serum or plasma) can be incubated.

By way of example, the antigen chip can have a size of 1.4 mm×2 mm. Conventionally, a positionally accurate application of antigen spots on a substrate surface is problematic. This requirement can be eliminated by embodiments of the present invention since the positioning of the antigen spots is determined within the method itself.

FIG. 6 illustrates an emission spectrum 83 of an illumination source 58 according to one embodiment of the present invention in exemplary fashion, wherein the wavelength is plotted along an abscissa 85 and the relative emission is plotted along an ordinate 87. The spectrum 83 exhibits an emission between 440 nm and 520 mm, with a maximum at approximately 470 nm. Such an emission spectrum can be used to produce excitation light 59 for exciting a first fluorescence and a second fluorescence.

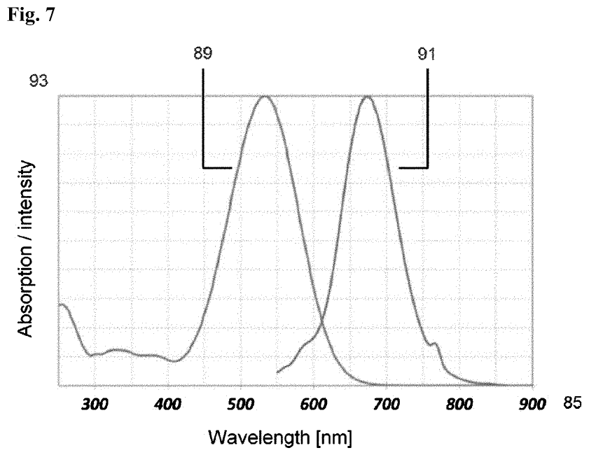

FIG. 7 illustrates an excitation spectrum 89 and an emission or fluorescence spectrum 91 of a first fluorescence dye, as can be used in embodiments of the present invention, with the wavelength being plotted on the abscissa 85 and the absorbance or emission intensity being plotted along the ordinate 93. Excitation is implemented above approximately 450 nm to approximately 605 nm, with a maximum at approximately 520 nm. The emission is implemented above approximately 555 nm to approximately 800 nm, with a maximum at approximately 670 nm. The example illustrated here in FIG. 7 is, the absorption and emission of the DY521XL dye (e.g., first fluorescence dye).

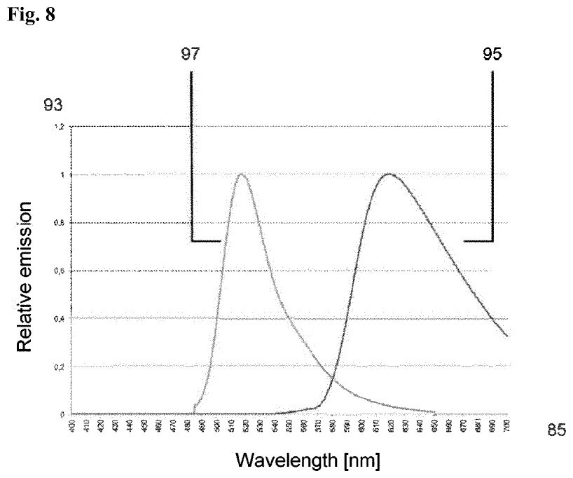

FIG. 8 illustrates emission or fluorescence spectra 95 and 97 of a first fluorescence dye and a second fluorescence dye in exemplary fashion, with the wavelength being plotted on the abscissa 85 and the emission intensity being plotted on the ordinate 93 again. As is evident from FIG. 8, the emissions of the first fluorescence 95 and of the second fluorescence 97 only overlap in a small region; i.e., they are specific for, firstly, detection of the spot locations and, secondly, detection of the bound secondary antibodies. By way of example, the fluorescence emission 97 can be realized by FITC; the fluorescence 95 can be realized, for example, by PI (propidium iodide).

Embodiments of the present invention can make do without a scanning process for recording the first images and second images. The magnification provided by the objective lens 69 may lie between 5, 8, 10, 15, 20 or 40 times, for example. According to embodiments of the present invention, approximately 300 pl of an antigen solution with a concentration of approximately 0.2 mg/ml suffices for each antigen spot. A patient sample of approximately 0.3 to 3 μl per field (i.e., six antigen chips) can be incubated in diluted fashion.

The present disclosure provides the following embodiments or subject matter:

1. An antigen chip (5) comprising:
a plane substrate surface (9);
antigen spots (11a, . . . , 11e), which are applied in a predetermined pattern on the substrate surface and which contain a first dye, a chromogenic substrate or a luminophore, in particular a first fluorescence dye.

2. The antigen chip according to the preceding embodiment, wherein antigen spots (11a, . . . , 11e), which contain different antigens, contain different amounts or concentrations of the first luminophore, chromogenic substrate or dye, in particular fluorescence dye, in such a way
that first radiation cast back therefrom, in particular induced first fluorescence, has substantially the same intensity or
that first radiation cast back by antigen spots of different lines, in particular induced first fluorescence, has substantially different intensities in order to be able to distinguish the lines.

3. The antigen chip according to any one of the preceding embodiments, wherein

(a) antigen spots (11a, . . . , 11e) of the same antigen are arranged in a line in each case, in particular with a predetermined spot spacing (d), wherein lines assigned to different antigens have, in particular, a predetermined line spacing (L) and/or arrangement,
wherein, further, the line spacing of two lines is greater than the spot spacing within a line, and/or
(b) antigen spots (11a, . . . , 11e) of the same antigen are arranged in a line in each case, wherein at least two lines have a distinguishable predetermined spot spacing (d).

4. The antigen chip according to any one of the preceding embodiments, wherein, further, at least two, in particular at least three, calibration spots (17) are applied to the substrate surface (9), said calibration spots containing a primary antibody, in each case in different amounts or concentrations.

5. The antigen chip according to any one of the preceding embodiments, wherein, further, at least one line (13) of an antigen, in particular a line with a greater or smaller dot spacing in comparison with the other lines of the antigen chip, is applied to the substrate as a positive control.

6. The antigen chip according to any one of the preceding embodiments, wherein

(a) the substrate surface (9) is microstructured for focusing purposes and, in particular, the substrate surface (9) has a size of between 1 mm×1 mm and 2 mm×4 mm, and/or
(b) the antigen chip has a cut-off calibrator.

7. An apparatus (50) for detecting antibodies in a sample, comprising:
an antigen chip (5) according to any one of the preceding embodiments; and
a scanner and/or camera system, in particular a fluorescence microscope (55) with:
   an illumination source (57), which is embodied to produce illumination light, in particular excitation light (59), and which is arranged to illuminate, in particular irradiate, the antigen chip (5);
   a first camera (61), which is embodied to record a first image (19) by detecting first radiation emanating from the antigen spots caused by the illumination light, in particular first fluorescence induced by the excitation light, and/or embodied to record a second image (77) by detecting chemiluminescence induced by the sample or second fluorescence induced by the illumination light, in particular excitation light,
      wherein the scanner and/or camera system, in particular the fluorescence microscope (55), further preferably comprises:
a second camera (65), which is embodied to record a second image (77) by detecting chemiluminescence induced by the sample or second fluorescence induced by the illumination light, in particular excitation light, in particular if the first camera is not embodied to detect the second fluorescence.

8. The apparatus according to embodiment 7, further comprising:
a first filter (62), which is arranged in a first beam path upstream of the first camera (61) and which is embodied to substantially eliminate excitation light and/or the light of the second fluorescence;
   in particular further comprising:
a second filter (66), which is arranged in a second beam path upstream of the second camera (65) and which is embodied to substantially eliminate excitation light and/or the light of the first fluorescence.

9. The apparatus according to either of embodiments 7 and 8, further comprising:
an evaluation system (75), which is embodied:
   to determine positions of the antigen spots on the basis of the first image;
   to assign positions of the antigen spots to respective antigens on the basis of the predetermined pattern; and/or
   to recognize antibodies bound to antigen spots, in particular at least semi-quantitatively, on the basis of the second image.

10. The apparatus according to any one of embodiments 7 to 9, wherein the evaluation system is further embodied:
to evaluate the second image in order to determine intensities of the second fluorescence emitted by the individual antigen spots and,
   in particular, to average intensities of the plurality of antigen spots containing the same antigen.

11. The apparatus according to any one of embodiments 7 to 10, wherein the evaluation system is further embodied:
to determine intensities of second fluorescence emanating from at least one, preferably at least two, in particular at least three calibration spot(s) (17) in the second image in order to carry out a calibration, wherein the calibration spots contain a primary antibody in different amounts or concentrations.

12. The apparatus according to any one of embodiments 7 to 11, wherein the evaluation system is further embodied:
to recognize at least one row of antigen spots (11a, . . . , 11e) and/or a line (13, 15), preferably at least one row and/or line with a greater and smaller dot spacing in comparison with the other rows and/or lines, particularly preferably two rows and/or lines with a greater and smaller dot spacing, in the first or second image, wherein a line and/or row is developed as a continuous line, in order to determine an orientation of the antigen chip and implement an assignment of the row and antigens to one another.

13. A method for producing an antigen chip (5) for indirect immunofluorescence diagnostics, including:
applying antigen spots (11a, . . . , 11e), which contain a first dye, chromogenic substrate or luminophore, in particular a first fluorescence dye, on a plane substrate surface (9) of a substrate (7) in a pattern that has been predetermined for each antigen,
fragmenting the substrate (7), in particular at arbitrary positions, in order to obtain a plurality of antigen chips (5).

14. The method according to embodiment 13, wherein the antigen spot is applied by means of a piezoelectric micro-metering appliance,

wherein, in particular, the substrate surface (9) is a surface of a glass substrate or of a glass substrate coated with a membrane and/or film.

15. A method for detecting antibodies in a sample, in particular by indirect immunofluorescence, wherein the method includes:
providing antigen spots (11a, . . . , 11b), which contain a first dye, chromogenic substrate or luminophore, in particular a first fluorescence dye, and which are arranged on a plane substrate (7) in a predetermined pattern;
incubating antigen spots with the sample;
incubating the antigen spots with a secondary antibody, which is marked by a second luminophore, second chromogenic substrate, enzyme or second fluorescence dye;
illuminating the antigen spots (11a, . . . , 11e) with illumination light, in particular excitation light (59), in order to trigger a first radiation, in particular in order to excite a first fluorescence (63) of the first fluorescence dye and, in particular, to excite a second fluorescence (67) of the second fluorescence dye;
detecting the first radiation, in particular the first fluorescence, by recording a first image (19) with a first camera (61) in order to determine positions of the antigen spots;
assigning the positions of the antigen spots to the respective antigens on the basis of the predetermined pattern; and
detecting the signals of the second luminophore, second chromogenic substrate or the chemiluminescence induced by the enzyme or the second fluorescence by recording a second image (77) with the first or a second camera (65) in order to recognize antibodies bound to antigen spots,
   wherein, in particular, an inaccuracy in the positioning of the antigen spots on the substrate (7) and/or the substrate relative to the first and/or the second camera is tolerated.

16. A kit for use in a method for detecting antibodies in a sample, in particular by indirect immunofluorescence, comprising:
an antigen chip according to any one of embodiments 1 to 6; and
a probe, in particular a secondary antibody, which is marked with a second fluorescence dye, luminophore, chromogenic substrate or enzyme,
   preferably, the first and the second dye each are a fluorescence dye and the first and the second fluorescence are excitable by way of the same wavelength.

1. An antigen chip comprising:
a plane substrate surface;
antigen spots, which are applied in a predetermined pattern on the substrate surface and which contain a first dye, a chromogenic substrate or a luminophore, in particular a first fluorescence dye.

2. The antigen chip according to claim 1, wherein antigen spots, which contain different antigens, contain different amounts or concentrations of the first luminophore, chromogenic substrate or dye, in particular fluorescence dye, in such a way
that first radiation cast back therefrom, in particular induced first fluorescence, has substantially the same intensity, or
that first radiation cast back by antigen spots of different lines, in particular induced first fluorescence, has substantially different intensities in order to be able to distinguish the lines.

3. The antigen chip according to claim 1, wherein
(a) antigen spots of the same antigen are arranged in a line in each case, in particular with a predetermined spot spacing (d), wherein lines assigned to different antigens have, in particular, a predetermined line spacing (L) and/or arrangement,
wherein, further, the line spacing of two lines is greater than the spot spacing within a line, and/or
(b) antigen spots of the same antigen are arranged in a line in each case, wherein at least two lines have a distinguishable predetermined spot spacing (d).

4. The antigen chip according to claim 1, wherein, further, at least two, in particular at least three, calibration spots are applied to the substrate surface, said calibration spots containing a primary antibody, in each case in different amounts or concentrations.

5. The antigen chip according to claim, wherein, further, at least one line of an antigen, in particular a line with a greater or smaller dot spacing in comparison with the other lines of the antigen chip, is applied to the substrate as a positive control.

6. The antigen chip according to claim 1, wherein
(a) the substrate surface is microstructured for focusing purposes and, in particular, the substrate surface has a size of between 1 mm×1 mm and 2 mm×4 mm, and/or
(b) the antigen chip has a cut-off calibrator.

7. An apparatus for detecting antibodies in a sample, comprising:
an antigen chip according to claim 1; and
a scanner and/or camera system, in particular a fluorescence microscope with:
an illumination source, which is embodied to produce illumination light, in particular excitation light, and which is arranged to illuminate, in particular irradiate, the antigen chip;
a first camera, which is embodied to record a first image by detecting first radiation emanating from the antigen spots caused by the illumination light, in particular first fluorescence induced by the excitation light, and/or embodied to record a second image by detecting chemiluminescence induced by the sample or second fluorescence induced by the illumination light, in particular excitation light,
wherein the scanner and/or camera system, in particular the fluorescence microscope, further preferably comprises:
a second camera, which is embodied to record a second image by detecting chemiluminescence induced by the sample or second fluorescence induced by the illumination light, in particular excitation light, in particular if the first camera is not embodied to detect the second fluorescence.

8. The apparatus according to claim 7, further comprising:
a first filter, which is arranged in a first beam path upstream of the first camera and which is embodied to substantially eliminate excitation light and/or the light of the second fluorescence;
in particular further comprising:
a second filter, which is arranged in a second beam path upstream of the second camera and which is embodied to substantially eliminate excitation light and/or the light of the first fluorescence.

9. The apparatus according to claim 7, further comprising:
an evaluation system, which is embodied:
to determine positions of the antigen spots on the basis of the first image;
to assign positions of the antigen spots to respective antigens on the basis of the predetermined pattern; and/or
to recognize antibodies bound to antigen spots, in particular at least semi-quantitatively, on the basis of the second image.

10. The apparatus according to claim 9, wherein the evaluation system is further embodied:
to evaluate the second image in order to determine intensities of the second fluorescence emitted by the individual antigen spots and,
in particular, to average intensities of the plurality of antigen spots containing the same antigen.

11. The apparatus according to claim 9, wherein the evaluation system is further embodied:
to determine intensities of second fluorescence emanating from at least one, preferably at least two, in particular at least three calibration spot(s) in the second image in order to carry out a calibration, wherein the calibration spots contain a primary antibody in different amounts or concentrations.

12. The Apparatus according to claim 9, wherein the evaluation system is further embodied:
to recognize at least one row of antigen spots and/or a line, preferably at least one row and/or line with a greater and smaller dot spacing in comparison with the other rows and/or lines, particularly preferably two rows and/or lines with a greater and smaller dot spacing, in the first or second image, wherein a line and/or row is developed as a continuous line, in order to determine an orientation of the antigen chip and implement an assignment of the row and antigens to one another.

13. A method for producing an antigen chip for indirect immunofluorescence diagnostics, including:
applying antigen spots, which contain a first dye, chromogenic substrate or luminophore, in particular a first fluorescence dye, on a plane substrate surface of a substrate in a pattern that has been predetermined for each antigen,
fragmenting the substrate, in particular at arbitrary positions, in order to obtain a plurality of antigen chips.

14. The method according to claim 13, wherein the antigen spot is applied by means of a piezoelectric micro-metering appliance,
wherein, in particular, the substrate surface is a surface of a glass substrate or of a glass substrate coated with a membrane and/or film.

15. A method for detecting antibodies in a sample, in particular by indirect immunofluorescence, wherein the method includes:
providing antigen spots, which contain a first dye, chromogenic substrate or luminophore, in particular a first fluorescence dye, and which are arranged on a plane substrate in a predetermined pattern;
incubating antigen spots with the sample;
incubating the antigen spots with a secondary antibody, which is marked by a second luminophore, second chromogenic substrate, enzyme or second fluorescence dye;
illuminating the antigen spots with illumination light, in particular excitation light, in order to trigger a first radiation, in particular in order to excite a first fluorescence of the first fluorescence dye and, in particular, to excite a second fluorescence of the second fluorescence dye;
detecting the first radiation, in particular the first fluorescence, by recording a first image with a first camera in order to determine positions of the antigen spots;
assigning the positions of the antigen spots to the respective antigens on the basis of the predetermined pattern; and
detecting the signals of the second luminophore, second chromogenic substrate or the chemiluminescence induced by the enzyme or the second fluorescence by recording a second image with the first or a second camera in order to recognize antibodies bound to antigen spots,
wherein, in particular, an inaccuracy in the positioning of the antigen spots on the substrate and/or the substrate relative to the first and/or the second camera is tolerated.

16. A kit for detecting antibodies in a sample, in particular by indirect immunofluorescence, comprising:
an antigen chip according to claim 1; and
a probe, in particular a secondary antibody, which is marked with a second fluorescence dye, luminophore, chromogenic substrate or enzyme,
preferably, the first and the second dye each are a fluorescence dye and the first and the second fluorescence are excitable by way of the same wavelength.
